SEQUENCE LISTING (4 sequences)

SEQ ID NO: 1 - DNA, 110 bp, Homo sapiens
caacttcagcaggctgtagtctgagaagcatcaattttcaacttcagcaggctgtgcagtcacaaggatttatcaattattgccaaaaaaaaattgatgcttctcagact

SEQ ID NO: 2 - DNA, 33 bp, Artificial Sequence
  synthetic DNA
cgcacttgccaaaaaaaattgatggtgcgtaaa

SEQ ID NO: 3 - DNA, 17 bp, Homo sapiens
caacttcagcaggctgt

SEQ ID NO: 4 - DNA, 21 bp, Homo sapiens
agtctgagaagcatcaatttt

(19) United States
(12) Patent Application Publication
(10) Pub. No.: US 2021/0005286 A1
(43) Pub. Date: Jan. 7, 2021

(54) METHODS FOR THE ANALYSIS OF DISSOCIATION MELT CURVE DATA

(71) Applicant: Biocartis NV, Mechelen (BE)

(72) Inventors: Bram De Craene, Mechelen (BE); Klaas Decanniere, Mechelen (BE); Jan Van De Velde, Mechelen (BE)

(21) Appl. No.: 16/767,967

(22) PCT Filed: Jan. 22, 2019
(86) PCT No.: PCT/EP2019/051511

§ 371 Date: May 28, 2020

(30) Foreign Application Priority Data
Jan. 23, 2018 (EP) 18153050.2

(51) Int. Cl. G16B 45/00; G16B 40/00; G16B 20/20

(52) U.S. Cl. CPC G16B 45/00; G16B 20/20; G16B 40/00

(57) ABSTRACT

The present invention relates to the use of wavelet transform to analyze raw melting curve data of nucleic acids. The effect is decreased noise sensitive calculations and increased computational efficiency and speed. The invention is particularly suitable for classifying test samples involving the combined analysis of several, multiplexed targets within one experiment generating large raw datasets requiring discrimination of minute variations in the data.

### FIELD OF THE INVENTION

The present application relates generally to the field of nucleic acid analysis. More in particular it applies to methods and systems that allow for the analysis of melting curve raw data and reliable interpretation of target nucleic acid information.

### BACKGROUND

There has been a great interest in developing molecular techniques to analyze nucleic acids, such as genomic DNA. Nucleic acid amplification methods are widely used for genomic analysis and permit quantitative analysis to determine nucleic acid copy number, sample source quantitation, and transcription analysis of gene expression.

Quantitative analysis comprises high-resolution melting or melt (HRM) curve analysis which is a versatile tool used to discriminate real amplification products from artifacts, for genotyping, and for variant scanning, especially suited for detecting small-scale variants such as simple sequence repeats or single base changes and when preceded by a nucleic acid amplification to provide a limited number or set of molecules with high abundance (Reed et al., 2007; Wittwer et al., 2009; Liao et al., 2013; Ramezanzadeh et al., 2016). One key characteristic in melting curve analysis is the melting temperature, Tm, the temperature at which 50% of the molecules of one specific duplex have dissociated. Often, melting curve analysis focuses on determining the Tm itself or Tm shifts as identified visually by the expert or using algorithms such as information maps, neural networks or smear detection algorithms (Palais et al., 2009). The reverse experiment can also be used; in that case one starts from dissociated molecules at high temperature, e.g. 95C, and one follows the association reaction as the temperature is gradually lowered. The melting profile of a PCR product depends on its GC content, length, sequence and heterozygosity, and vastly different molecules can have similar melting temperatures. Melting curve analysis is therefore usually invoked to discriminate between small-scale variant. These small-scale variants cannot be resolved with amplification-only experiments.

Most, if not all, modem implementations measure change of fluorescence as a function of temperature at a specific wavelength band, although more intricate methods have been devised (Gray et al., 2011). Change of fluorescence can be obtained by using intercalating dyes that co-dissociate during melting or via interactions with specific molecular reporters called molecular beacons. The raw measurements need processing, either by hand or by using a computer program, to characterize and identify the various oligonucleotides in the mixture under investigation. Data processing usually starts with background removal and then focuses either on identifying Tm differences or on curve shape differences between the sample curve and some reference signal. The reference signal is usually obtained either from one well known oligonucleotide, from a mixture of well-characterized oligonucleotides or by calculations starting from sequence information. Usually, a method is applied where the derivative curve of the raw data is calculated. The graph of the negative first derivative of the melting curve makes it easier to pinpoint the temperature of dissociation by the virtue of peaks thus formed. Various algorithms exist to obtain the derivative curve. Several approaches also exist for identifying “significant” peaks or peak shifts. Peak position, peak height and sometimes also peak width are uses as features in further analysis. Signals can easily be analyzed using Fourier transforms, a powerful mathematical tool important in signal processing that analyzes what frequencies are present in a signal and in what proportions.

Methods and optimization methods in the field of melting curve analysis of nucleic acids have been described.

EP2241990 describes a method where a double sigmoid of the form

$$f ( x ) = a + b x + \frac{c}{( 1 + {exp }^{- d ( x - e )} ) ( 1 + {exp }^{- f ( x - g )} )}$$

is fitted to the measured data. Subsequently, a derivative is obtained analytically from this fitted curve, and the Tm is obtained by determining a maximum in the derivative curve.

U.S. Pat. No. 6,106,777 describes a method for single-stranded DNA fragments where the melting curve for an unknown sample is compared with a collection of melting curves measured for known DNA fragments. The known curve or combination of curves with the smallest statistical error vs the “unknown” curve is then considered representative for that unknown sample.

U.S. Pat. No. 8,068,992 describes a method for melting curve background correction using a decreasing exponential.

EP2695951 describes methods for Tm determination for a cluster, where the Tm is determined by finding a peak in the negative first derivative curve or applying a threshold to a normalized melting curve.

U.S. Pat. No. 9,273,346 described a method that determines a deviation function capturing the difference between the sample measurements and a mathematical model describing the expected background for a blank measurement run. This deviation curve is the further analysed. US201400067345 describes a method where the measured data is noise-corrected, scaled, and fitted to an estimated asymptote for a low temperature region and finally clustered.

Patent EP2226390 describes a method where pre-determined “high” and “low” temperature ranges Th and Tl are defined, a signal difference representative for a molten state is identified, and the highest signal differential value observed is selected as a first peak candidate. This peak candidate is checked by confirming that the temperature linked to that signal differential is either within the Th or the T range, and that there is no peak candidate outside these temperature ranges.

US20050255483 proposes a method to smooth melting curve data based on a collapse number, a method somewhat similar to calculating a moving average. The smoothed data can then be used for further processing, including derivative calculations.

Patent WO2017025589 defines a method for the analysis of melting curves following a PCR reaction where the negative slope is calculated at each raw data point to generate the melting curve. This melting curve is then subject to spectral analysis using Fourier analysis methods, with the aim of extracting features suitable for classification algorithms such as SVM, LVQ or Random Forrest which are used to show the presence of a specific nucleic acid and/or to determine the amount present in the sample.

Athamanolap et al. (2014) describe a feature engineering post-processing step of the raw data prior to classification of the melting curves with machine learning algorithms. In this post-processing step the initial set of measurement values is interpolated through piecewise linear interpolation to a set of 300 values. In contrast to traditional melt curve analysis the temperature values are chosen as the dependent variable. This set is again interpolated to obtain 1000 data points and this data vector is analysed using machine learning algorithms.

While many applications exist for HRM analysis, discrimination at the single nucleotide remains challenging, due to the minute Tm shifts that must be detected.

One limitation of current methods is related to the calculations applied to obtain the first derivative curve from the raw data. These calculations are sensitive to noise, requiring either some form of smoothing or a way to differentiate between the “true” peaks formed and peaks introduced or “enhanced” by the noise.

A second limitation concerns sub-optimal capture of all information present in the data. Most often, peak search and Tm identification only captures part of the information.

Alternatively, the described “curve shape” methods do capture all information but this approach results in large feature vectors and subsequently cumbersome further processing or classification, which provides a further limitation of the current methods.

Accordingly, there appears to be a need in the art for improved methods of analyzing small differences in melt or melting curves in the presence of the inherent noise of the analysis.

It is an objective of the present invention to remedy all or part of the disadvantages mentioned above.

The present invention fulfills these objectives by providing methods that use wavelet transform to analyse raw, i.e. non transformed by any mathematical function, melting curve data. The effect is decreased noise in sensitive calculations and increased computational efficiency and speed. In methods of present invention it is important that the raw fluorescence melting curve data readings as collected in function of changing temperature are not transformed mathematically or otherwise changed before being subjected to the wavelet transform. In other words, it is essential that the wavelet transform is applied directly on the raw melting curve data as collected throughout the entire raw data collection process or during a selected part thereof or a window within it (i.e. performed on a continuous selection of raw data as captured during the raw melting curve data collection process). This means that between the collecting of the raw melting curve data and generating its wavelet-transformed version, the methods of the invention do not perform any other mathematical data transformation like computing a derivative, interpolation, resampling, oversampling etc. The only operation that present methods may involve before applying the wavelet transform on the raw data is choosing a selection in the whole raw data as collected, e.g. from temperature point 1 (T1) to temperature point 2 (T2), and then applying the wavelet transform only on this specific selection of raw data window from T1 to T2. By making such selection, the amount of raw data that has to be processed via the wavelet transform is reduced, which is advantageous for the speed of performing computations, but is by no means modified in any mathematical way so the sensitivity of the method performed within said raw data window from T1 to T2 is preserved.

There exist very few teachings of applying wavelet transform to fluorescence readings in the field. However, none of them involves a use of the wavelet transform to analyse raw, i.e. non transformed, melting curve data. For example, US20090037117 generally teaches methods of transforming raw fluorescence emission data as collected to generate improved first-order or other derivative plots. However, although US20090037117 mentions a use of a frequency transform that can comprise a wavelet transform (mentioned among many other existing transform types), it explicitly teaches that prior to being subjected to such transform, the raw data has to be interpolated, oversampled, or resampled to produce data points at equally-spaced temperature intervals. Therefore, US20090037117 never teaches or suggests use of a wavelet transform on raw melting curve data.

Another example is, CN102880812 that mentions processing of a melting curve based on a wavelet analysis method but in the method of CN102880812 the fluorescence signal is first plotted as a first derivative and the following mathematical transformations only start from the first derivative of the data. Consequently, also CN102880812 does not teach the benefits of applying wavelet transform on raw melting curve data.

Lastly, CN103593659 teaches use of a wavelet for analyzing peaks in a chromatogram from a Sanger sequencing reaction. Therefore, CN103593659 does not teach application of a wavelet transform to raw melting curve data. Furthermore, CN103593659 also explicitly teaches that the chromatogram data has to be filtered and denoised.

Consequently, the method of the invention comprising performing discrete wavelet transform on raw fluorescence read-out (or a selected part thereof) obtained from melting curve nucleic acid analysis has never been disclosed in the art. The methods of the invention are particularly suitable for classifying test samples involving the combined analysis of several, multiplexed targets within one experiment generating large raw datasets requiring discrimination of minute variations in the data. Their main advantages involve decreasing of noise and improving computational efficiency and speed. These and other advantages of the invention are explained in the continuation.

### SUMMARY OF THE INVENTION

In one embodiment, the present invention provides a method for analyzing melting curve raw data of nucleic acid from a test sample, the method comprising the steps of:
producing melting curve raw data from a nucleic acid;
performing discrete wavelet transform on the raw data to produce dwt coefficients;
performing the analysis of the dwt coefficients;

and classifying the test sample based on the analysis.

In a related aspect, a method for analyzing melting curve raw data of nucleic acid from a test sample is provided wherein the steps of:
producing melting curve raw data from a nucleic acid;
performing discrete wavelet transform on the raw data to produce dwt coefficients;
performing the analysis of the dwt coefficients; and
classifying the test sample based on the analysis

are performed in an automated system.

Another aspect relates to a computer-implemented method for obtaining and transforming melting curve raw metrics of nucleic acid from a test sample, the method comprising the steps of:
producing melting curve raw data from a nucleic acid;
performing discrete wavelet transform on the raw data to produce dwt coefficients;
selecting those dwt coefficients identified as most relevant for analysis of said nucleic acid;
performing the analysis of the selected dwt coefficients; and
classifying the test sample based on the analysis.

The invention further relates to a data processing device comprising means for carrying out the computer-implemented method for obtaining and transforming melting curve raw data of nucleic acid from a test sample.

It also relates to a computer program comprising instructions which, when the program is executed by a computer, cause the computer to carry out the computer-implemented method for obtaining and transforming melting curve raw data of nucleic acid from a test sample.

It also relates to a computer-readable medium comprising instructions which, when executed by a computer, cause the computer to carry out the computer-implemented method for obtaining and transforming melting curve raw data of nucleic acid from a test sample.

### BRIEF DESCRIPTION OF THE DRAWINGS

FIG. 1: Flow chart of an example method for analyzing melting curve raw data of nucleic acid from a test sample.

FIG. 2: Graphs representing raw melting profile of the SEC31A gene in a reference sample in function of temperature. Measurement of fluorescence is represented on the Y-axis; the measured melting cycles are indicated on the X-axis. One fluorescence measurement is taken per 0.3° C. temperature increase. Each curve represents one melting profile of the SEC31A gene in a reference sample. Data is shown for 317 samples, illustrating the variability in data measurements.

FIG. 2A: melting profile shown by dashed lines with squares representing samples that are characterized as 20% mutated+80% WT (MSI).

FIG. 2B: melting profile shown by full lines with crosses representing samples that are characterized as 100% WT (MSS).

FIG. 2C: melting profile shown by dotted lines with circles representing samples that are characterized as empty sample (NTC), showing the melting profile of the hairpin structure of the molecular beacon.

FIG. 3: Graphs representing a set of dwt coefficients for the SEC31A gene using the scale function from Daubechies DB8. The coefficients in the third level of decomposition are displayed. Data is shown for 317 samples.

FIG. 3A: dashed line with squares representing samples that are characterized as 20% mutated+80% WT (MSI).

FIG. 3B: full lines with crosses representing 100% WT (MSS).

FIG. 3C: dotted lines with circles representing empty sample (NTC).

FIG. 4: Graphs representing a set of dwt coefficients for the SEC31A gene using the wavelet function from Daubechies DB8. The coefficients in the third level of decomposition are displayed. Data is shown for 317 samples.

FIG. 4A: dashed line with squares represents samples that are characterized as 20% mutated+80% WT (MSI).

FIG. 4B: full lines with crosses are 100% WT (MSS).

FIG. 4C: dotted lines with circles are empty sample (NTC).

FIG. 5: Graph representing one set of dwt coefficients using the scale function from Daubechies DB8 for each of the three main classes of samples. The coefficients in the third level of the decomposition are displayed. Each curve represents one wavelet profile of the SEC31A gene in a reference sample. The dashed line with squares represents a sample that is characterized as 20% mutated+80% WT (MSI), the full line with crosses is 100% WT (MSS) and the dotted line with circles is an empty sample (NTC). The figure emphasizes the differences in the scale function patterns obtained for the three classes of samples.

FIG. 6: Graph representing one set of dwt coefficients using the wavelet function from Daubechies DB8 for each of the three main classes of samples. The coefficients in the third level of the decomposition are displayed. Each curve represents one wavelet profile of the SEC31A gene in a reference sample. The dashed line with squares represents a sample that is characterized as 20% mutated+80% WT (MSI), the full line with crosses is 100% WT (MSS) and the dotted line with circles is an empty sample (NTC). The figure emphasizes the differences in the scale function patterns obtained for the three classes of samples.

FIG. 7: Graphs representing a set of dwt coefficients for the SEC31A gene using the scale function from Daubechies DB4. The coefficients in the third level of decomposition are displayed. Data is shown for 317 samples.

FIG. 7A: dashed line with squares representing samples that are characterized as 20% mutated+80% WT (MSI).

FIG. 7B: full lines with crosses representing 100% WT (MSS).

FIG. 7C: dotted lines with circles representing empty sample (NTC).

FIG. 8: Graphs representing a set of dwt coefficients for the SEC31A gene using the wavelet function from Daubechies DB4. The coefficients in the third level of decomposition are displayed. Data is shown for 317 samples.

FIG. 8A: dashed line with squares representing samples that are characterized as 20% mutated+80% WT (MSI).

FIG. 8B: full lines with crosses representing 100% WT (MSS).

FIG. 8C: dotted lines with circles representing empty sample (NTC).

FIG. 9: Graphs representing a set of dwt coefficients for the SEC31A gene using the scale function from the Haar wavelet. The coefficients in the third level of decomposition are displayed. Data is shown for 317 samples.

FIG. 9A: dashed line with squares representing samples that are characterized as 20% mutated+80% WT (MSI).

FIG. 9B: full lines with crosses representing 100% WT (MSS).

FIG. 9C: dotted lines with circles representing empty sample (NTC).

FIG. 10: Graphs representing a set of dwt coefficients for the SEC31A gene using the wavelet function from the Haar wavelet. The coefficients in the third level of decomposition are displayed. Data is shown for 317 samples.

FIG. 10A: dashed line with squares representing samples that are characterized as 20% mutated+80% WT (MSI).

FIG. 10B: full lines with crosses representing 100% WT (MSS).

FIG. 10C: dotted lines with circles representing empty sample (NTC).

### DETAILED DESCRIPTION OF THE INVENTION

The invention can be implemented in numerous ways, including as a process or method; an apparatus; a system; a computer program method or product, a computer program, a computer readable storage medium and/or a processor, such as a processor configured to execute instructions stored on and/or provided by a memory coupled to the processor. In this specification, these implementations, or any other form that the invention may take, may be referred to as methods. In general, the order of the steps of disclosed methods may be altered within the scope of the invention.

As used herein, the term “or” is an inclusive “or” operator and is equivalent to the term “and/or” unless the context clearly dictates otherwise. The meaning of “a”, “an”, and “the” include plural references.

As used herein, the term “DWT” designates discrete wavelet transform; the term “dwt coefficient” designates discrete wavelet transform coefficient. A wavelet transform means a calculation using a program or subroutine on raw data. Thus a set of dwt coefficients is a discrete wavelet transformed set of values. The most relevant dwt coefficients for nucleic acid analyses are those coefficients that capture the significant events of the experiment, for example in case of a melting experiment of a double-stranded nucleic acid molecule the most relevant dwt coefficients can be peaks or peak shifts in the raw data melting curves.

As used herein, the terms “melting curve raw data”, “raw data melting curve” and “raw melting curve data” are equivalent and used interchangeably. As used herein, they are meant to be interpreted as referring to an unmodified (“raw”) set of numerical values captured from fluorescence measurements performed during nucleic acid dissociation or association experiment in function of changing temperature (i.e. fluorescence measurements performed during a melting curve experiment). In other words, it can be stated that they designate machine-captured florescence signal-associated identifiers obtained following nucleic acid dissociation or association experiments that are not mathematically transformed or modified by any function

As used herein, the term “performing discrete wavelet transform on the raw data” is to be interpreted as referring to performing discrete wavelet transform directly on an unmodified set of numerical values collected from a melting curve experiment. The term “unmodified” as used herein means not transformed mathematically or otherwise changed by any mathematical value-transforming function before being subjected to the wavelet transform. This means, that within the ambit of present invention, the wavelet transform is applied directly on the raw melting curve data as collected throughout the entire raw data collection process or during a selected part or within a window selected during this collection process. This means that between the collecting of the raw melting curve data and generating its wavelet-transformed version, the methods of the invention do not perform any mathematical data transformation including e.g. computing a derivative, interpolation, resampling, oversampling etc. The only operation that present methods may involve before applying the wavelet transform on the raw data is choosing a selection (or as sometimes used herein, a “window”) in the whole raw data set as collected from e.g. temperature point 1 (T1) to temperature point 2 (T2). In this example, once the entire data raw set is reduced by ignoring raw data values outside of said window, the wavelet transform is applied on only the specific selection of raw unmodified data as encompassed within said selected window from T1 to T2. By making such selection, the amount of raw data that has to be processed via the wavelet transform is reduced, which is advantageous for the speed of performing computations. In line with the above, as used herein, the expression “performing data reduction on the raw data to generate a selection of raw data” is to be interpreted as selecting a continuous set of non-modified raw data values from a window comprised in the entire set of all raw data values as collected during a melting curve experiment, and ignoring the non-modified raw data values from outside of said window. One possible reason of ignoring such raw data values from outside the selected raw data window would be because they will not comprise any valuable information related to characterization of a given nucleic acid, for example they would comprise raw florescence data below or very close to detection threshold etc. Therefore, as used herein, the term “data reduction” is to be interpreted as merely referring to a selection of raw data in a preferred, possibly information-rich window within the entire raw data set as collected, and should by no means imply application of any mathematical value-transforming function comprising reduction operation, since the raw data values comprised in the selected window in disclosed herein methods remain intact.

It is an aspect of the present invention to provide methods of improved target nucleic acid analysis. The methods may be part of a complete service and product, including amplifying parts of a subject's genome; obtaining melting curve raw data of the amplified parts; amplifying multiple parts of a subject's genome concurrently; using multiple reactions vessels concurrently for said amplification; measuring multiple independent reporter molecules within one reaction; discriminating between said multiple reporter molecules using color filters; discriminating between multiple reporter molecules using a color-sensitive detector; data processing by discrete wavelet transform; use of all obtained wavelet coefficients to classify a test sample; use of some of the obtained wavelet coefficients alone or in combination with other features to classify a test sample; storage of the data and coefficients; and reporting.

In one embodiment, the present invention provides a method for analyzing melting curve raw data of nucleic acid from a test sample, the method comprising the steps of:
producing melting curve raw data from a nucleic acid;
performing discrete wavelet transform on the raw data to produce dwt coefficients;
performing the analysis of the dwt coefficients;

and classifying the test sample based on the analysis.

A particular embodiment of the present invention concerns a method for analyzing melting curve raw data of nucleic acid from a test sample, the method comprising the steps of:
providing a source of nucleic acid from a subject;
amplifying said nucleic acid;
dissociating or associating the amplified nucleic acid to produce melting curve raw data;
optionally, performing data reduction on the raw data to generate a selection of raw data;
performing discrete wavelet transform on the selection of raw data to produce dwt coefficients;
performing the analysis of the dwt coefficients;

and classifying the test sample based on the analysis.

Typically, the source of nucleic acid potentially comprises a target sequence under investigation.

In a particular embodiment, said method is preceded by any of the following steps of:
liberating and/or isolating the nucleic acid potentially comprising the target sequence from the source of a nucleic acid;
providing said liberated and/or purified nucleic acid potentially comprising the target to the step of amplifying said nucleic acid.

The nucleic acid for use in the methods of the invention can be naturally existing, modified or artificial nucleic acid. In a preferred embodiment, the process of the invention starts with providing a source of nucleic acid. The nucleic acid under investigation is derived from a human or animal subject, preferably from a patient sample. The biological sample comprises nucleic acids or cells comprising nucleic acid to be analyzed according to the methods of the invention. The sample may be a tissue sample, swab specimen, body fluid, body fluid precipitate or lavage specimen. Non-limiting examples include human or animal fresh tissue samples, frozen tissue samples, tissue samples embedded in FFPE (formalin-fixed paraffin-embedded tissue), whole blood, blood plasma, blood serum, urine, stool, saliva, cerebrospinal fluid, peritoneal fluid, pleural fluid, lymph fluid, nipple aspirate, sputum and ejaculate. The samples may be collected using any suitable methods known in the art.

Methods and systems for obtaining nucleic acid from samples have been described and may require isolation and/or purification of the nucleic acid from the sample or liquefaction of the sample to liberate the nucleic acid under investigation (WO2014128129), or a combination thereof. In a particular aspect, the sample is obtained from a patient with suspected gastrointestinal malignancies, such as colon, colorectal or gastric cancer.

As used herein, the term “nucleic acid” and its equivalent “polynucleotide”, refer to a polymer of ribonucleosides or deoxyribonucleosides comprising phosphodiester linkages between nucleotide subunits.

The nucleic acid molecule to be analyzed include DNA or RNA, such as genomic DNA, mitochondrial or meDNA, cDNA, mRNA, tRNA, hnRNA, microRNA, IncRNA, siRNA, etc. . . . or any combination thereof. Typically parts of the nucleic acid molecules are amplified before melting curve analysis. Typically amplification uses polymerase chain reaction or PCR, preferably qualitative PCR (qPCR). The key feature of qPCR is that the nucleic acid product is being detected during thermocycling as the reaction progresses in “real time”. Remark, the double stranded nucleic acid molecules can also be non-amplified double stranded molecules. This is possible if the nucleic acid content of the sample is sufficiently high to allow detection. Thus, the amplification step may be an optional step in the methods & systems of the invention. Single stranded nucleic acids can in turn be analyzed after amplification reaction or hybridization with a second nucleic acid resulting in a double stranded structure. For RNA analysis, the amplification step is typically preceded by a reverse transcription (RT) step.

Thus, the present methods concern the detection of changes in target nucleotides sequence or nucleotide numbers in a nucleic acid. They may require discrimination at the single nucleotide level. In a preferred setting of the methods, amplicons are hereto generated by amplifying part of a nucleic acid sequence, said part comprising the particular target sequence under investigation. Minimum necessary arrangement of reagents and elements for performing a qPCR usually include any reagents allowing detection in real time PCR thermocycling of a nucleic acid template, e.g. DNA received from a source of nucleic acid. Such reagents include but depending on the type of qPCR are not limited to a PCR-grade polymerase, at least one primer set, a detectable dye or a probe, dNTPs, PCR buffer etc. Those skilled in the art will recognize that other techniques can be used to amplify nucleic acids.

Melting or melt curve analysis is an assessment of the dissociation or association-characteristics of a double-stranded nucleic acid molecule during temperature variation. As used herein, melting curve data concern data representing either dissociation or association characteristics of the nucleic acid molecule under investigation.

Melting curve analysis and HRM (high resolution melting) analysis are commonly used methods for detecting and analyzing the presence of nucleic acid sequences in a sample. One way of monitoring dissociation and association characteristics of a nucleic acid happens with the aid of dyes. The detection chemistries used for qPCR and melt curve analysis rely on (a) chemistries that usually detect fluorescence of a target-binding dye, e.g. a DNA-binding fluorophore such as LC Green, LC Green+, Eva Green, SYTO9 CYBR Green, or (b) target specific chemistries that usually utilize fluorophore-labeled DNA probes, such as e.g. beacon probes, and/or primers, such as e.g. scorpion primers. It is well known in the art that other detection chemistries can be applied in melt curve analysis Fluorophores absorb light energy at one wavelength and, in response, re-emit light energy at another, longer wavelength. Each fluorophore has a distinctive range of wavelengths at which it absorbs light and another distinct range of wavelengths at which it emits light. This property enables their use for specific detection of amplification products by real-time PCR instruments and by other analysis tools and/or analysis techniques. The same property allows observing different fluorophores within one reaction using color filters if their absorption and re-emission wavelength bands are non-overlapping (multiplexing). Thus, combinations of fluorophores allow for the detection of a range of amplification products or for multiplexing. Fluorophores can eventually be used in combination with quencher molecules, the quencher quenches the fluorescent emission of the fluorophore such that no signal is generated. Removal of the quencher from the fluorophore results in the generation of the fluorescent signal. Detection methods involving quenchers and quenchers applicable in such methods have been described and are well known in the art.

Accordingly, one embodiment of the present invention involves dissociation measurements. In one particular embodiment, the nucleic acid, e.g. DNA, is heated in the presence of one or more intercalating dyes during a melting curve test procedure. The dissociation of the DNA during heating is measurable by the large reduction in fluorescence that results. In another particular embodiment, the nucleic acid, e.g. DNA, is heated in the presence of one or more dye-labeled nucleic acid, e.g. one or more probes, during a melting curve test procedure. In case of probe-based fluorescence melting curve analysis variation detection in nucleic acids is based on melting temperature generated by thermal denaturation of the probe-target hybrid. As the heating of the nucleic acid, or the generated amplicons in case of amplification, proceeds, the changes in the strength of the signal are detected in the function of temperature, typically over a temperature interval, to obtain melting curve raw data.

As discussed and shown in the example section, the melting curve raw data are preferably generated with the aid of target specific chemistries that usually utilize fluorophore-labeled DNA probes, in particular molecular beacon probes. In principle, in possible embodiments, any target-specific oligonucleotide probe suitable for performing melting curve analysis can be used in the method of the invention. Preferred known probes may comprise a pair consisting of a fluorophore and a quencher, and may also advantageously form secondary structures such as loops or hairpins. The molecular beacon probes, or molecular beacons, are hairpin shaped molecules with an internally quenched fluorophore whose fluorescence is restored when they bind to a target nucleic acid sequence. For this reason, molecular beacons are not degraded by the action of polymerase and can be employed in studying their hybridization kinetics to their target via melting curve calling. The structure and working mechanism of molecular beacons is well known in the art. A typical molecular beacon probe is about 25 nucleotides long or longer. Typically, the region that is complementary to and binds to the target sequence is a 18-30 basepair region.

Nucleic acid variation detection may happen at the single nucleotide level and may involve detection of single nucleotide mutations, single nucleotide insertions or deletions. A practical example where melt curve analysis in accordance with embodiments of the invention may be used is a typical single nucleotide polymorphism (SNP), single nucleotide insertion or deletion detection scenario where the sample under study can be either homozygous or heterozygous for the SNP, insertion or deletion of interest.

Accordingly, current invention provides in a particular specific setting a method for analyzing the melting profile obtained from nucleic acids with one or more SNP's, insertions or deletions. To that purpose, the method of the present invention uses dye-labeled probes, to detect one or more SNP's, insertions or deletions in a standard quantitative PCR thermocycling instrument without the need for any additional equipment for post-PCR analysis. Thus, in a particularly advantageous embodiment, the signal-generating reagent is at least one labeled (i.e. signal-generating) oligonucleotide probe, preferably being a molecular beacon probe, comprising a sequence complementary to the sequence of one or more target SPN's, insertion or deletion and capable of hybridizing to said target sequence. Most preferably, the sequence capable of hybridizing to the target sequence comprises a sequence identical to or perfectly complementary to a mutant of said target sequence, said mutant comprising one or more nucleotide variations as compared to its wild-type form. Variance is then measured between the raw melting data of the wild type and the mutant and will be characteristic of the melting curve raw data.

As shown in the example section, the nucleic acid targeted for amplification and melting curve analysis will in a particular setting be associated with microsatellite instability (MSI). MSI is due to defective mismatch repair. Microsatellite sequences associated with human gastrointestinal cancer, particularly colorectal cancer and their analysis using fluorophore-labeled probes have been described in WO2013153130 and WO2017050934. An MSI screening test looks for changes in the DNA sequence between normal tissue and tumor tissue and can identify whether or not there is high amount of instability, which is called MSI-High (MSH). The opposite of MSH is called MSS, which stands for Microsatellite Stable.

Thus, current invention further provides in a particular specific setting a method for analyzing the melting profile obtained from nucleic acids with subtle microsatellite changes. To that purpose, the method of the present invention uses dye-labeled probes, to detect length variations in the short homopolymeric repeat regions in a standard quantitative PCR thermocycling instrument without the need for any additional equipment for post-PCR analysis. Thus, in a particularly advantageous embodiment, the signal-generating reagent is at least one labeled (i.e. signal-generating) oligonucleotide probe, preferably being a molecular beacon probe, comprising a sequence complementary to the target homopolymeric repeat sequence and capable of hybridizing to said target homopolymeric repeat sequence and its specific flanking sequence. Most preferably, the sequence capable of hybridizing to the target homopolymeric repeat sequence comprises a sequence identical to or perfectly complementary to a mutant of said target homopolymeric repeat sequence, said mutant comprising a deletion of at least one homonucleotide in said target homopolymeric repeat sequence as compared to its wild-type form. Variance is then measured between the raw melting data of the wild type and the mutant and will be characteristic of the melting curve raw data.

The temperature interval used to obtain melting curve raw data is chosen such that the dissociation event is observed. Typically, the melting temperature for the double strand nucleic acid has to be enclosed in the temperature interval such that the strands dissociate and the dye is released. Alternatively, the temperature is chosen such that full dissociation of the probe is achieved. The methods of the invention aim at detecting small-scale variants such as single nucleotide variations, such as single nucleotide mutations, single nucleotide insertions or single nucleotide deletions. Therefore, the temperature increments have to be small, i.e., at least smaller than 5° C. Even better if they are smaller than 4° C., 3° C., 2° C., or 1° C. Typically each temperature increment within a chosen interval is smaller than 0.5° C., equal or smaller than 0.4° C., preferably equal or smaller than 0.3° C., possibly equal or smaller than 0.2° C. or in some applications even equal or smaller than 0.1° C. (or interval equal to the smallest temperature error that can be maintained by the device). In a particular setting of the methods, the fluorescence is measured for each temperature increase step. In case multiplexing is applied, fluorescence is measured for each temperature increase step for each fluorophore.

In an example using multiplexing for instance, the temperature range for the experiment may be chosen such that full dissociation of each probe is ensured, and dissociation of each individual probe may be fully characterized by a smaller temperature interval. However, in particular settings of the experiment, the temperature increment within a chosen interval may be chosen too small resulting in the measurement of redundant data. This redundant data may then be removed from the raw data set. In such case, for instance, every second or every third measurement is removed from the raw data set without loosing the information relevant for the further analysis. This is particularly beneficial in case multiplexing is applied and larger raw data sets are generated.

Accordingly, current invention further provides in a particular specific setting a method for analyzing the melting profile obtained from nucleic acids, wherein the step of producing melting curve raw data from a nucleic acid is followed by a step of performing reduction on the raw data to generate a selection of raw data. In a particularly advantageous embodiment the data reduction step involves removal of redundant data from the raw data, preferably removal of measurements is applied at a repeated frequency. If data reduction is applied, it is immediately followed by the step performing a discrete wavelet transform (DWT) on the selection of the raw data. If data reduction is not applied, a DWT is generated directly from the raw data.

In a further step, transformations are applied to obtain further information from that data that is not readily available in the raw data set. Thus, transformations extract useful information embedded in the raw data. Prior art methods applying the conversion of raw nucleic acid melting data to derivative curves often involve the amplification of background noise and artificial smoothing of significant features of the melting data. The methods of the present invention apply a discrete wavelet transform (DWT) calculation directly on the raw metrics or directly on a reduced set of raw metrics obtained by a process of dissociation of double stranded nucleic acid during heating. By doing so, noise-sensitive derivative calculations of the raw data are avoided. The present methods are particularly suitable for distinguishing subtle but molecularly significant differences in raw nucleic acid melting data, which is an advantage over previous techniques that involved derivative curve analysis.

Wavelets are mathematical functions that cut up data into different frequency components, and then study each component with a resolution matched to its scale. These basis functions are short waves with limited duration. The basis functions of the wavelet transform are scaled with respect to frequency. There are many different wavelets that can be used as basis functions. The basis function ˜(t), also called the mother wavelet is the transforming function.

The term wavelet means a small wave. The smallness refers to the condition that this (window) function is of finite length (compactly supported). The wave refers to the condition that this function is oscillatory. The term mother implies that the functions with different region of support that are used in the transformation process are derived from one main function, or the mother wavelet. In other words, the mother wavelet is a prototype for generating the other window functions. In general, the wavelet ψ(t) is a complex valued function. A general wavelet function is defined as:

ψs,τ(t)=|s|^{-1/2}ψ[(t-τ)/s]

ψs,τ(t)=|s|^{-1/2}ψ[(t-τ)/s]

This shift parameter ‘τ’ determines the position of the window in time and thus defines which part of the signal x(t) is being analyzed. In wavelet transform analysis, frequency variable ‘ω’ is replaced by scale variable ‘s’ and time shift variable ‘t1’ is replaced by ‘τ’.

The Wavelet Transform utilizes these mother wavelet functions, and performs the decomposition of the signal x(t) into weighted set of scaled wavelet functions ψ(t). The main advantage of using wavelets is that they are localized in space.

A DWT is any wavelet transform for which the wavelets are discretely sampled. As with other wavelet transforms, a key advantage it has over Fourier transforms is temporal resolution: it captures both frequency and location information (location in time). Application of the wavelet transform on the raw metrics produces a set of reconstruction output wavelet coefficients at different scales (a) one is the approximation output which is the low frequency content of the input signal component and (b) the other is the multidimensional output which gives the high frequency components, being the details of the input signal at various levels. This separation of features into different scales (or frequencies) allows for an operator or computer algorithm to select the wavelet coefficients most relevant for certain decisions or analysis, a process often referred to as wavelet filtering. This process can be applied repeatedly, splitting up the signal in multiple frequency bands. When applied on melting curve data, the highest frequency wavelet coefficients are mostly noise whereas the lowest resolution coefficients capture information related to instrument gain or amplification efficiency in the preceding amplification reaction. Both have little or no relevance for the identification of a specific oligonucleotide in a sample subject to melting curve analysis itself but potentially have relevance with respect to reliability of such identification. Packages containing all functions necessary for computing and plotting discrete wavelet transforms (DWT) have been described (Aldrich, 2015).

As shown in the example, the step of performing discrete wavelet transform on the data to produce discrete wavelet transform coefficients (dwt coefficients) will in a particular setting calculate a one-dimension (D) wavelet transform of the raw data or the reduction data using a mother wavelet from the Daubechies family. The mother wavelet is the unmodified wavelet chosen as basis for the discrete wavelet transform (Daubechies, 1992). Good results were obtained when the DB8 mother wavelet was used. The mother wavelet is subsequently dilated, shifted and scaled, using the pyramid dwt algorithm, to generate a set of child wavelets that best represent the signal to be analyzed; the set of wavelet and scale coefficients obtained from the algorithm being the result of the discrete wavelet transform. In the specified example, boundary conditions for the DWT are periodic. The raw data input to the transform can be all data measured or a subset that covers all significant events of the experiment.

Thus, one step in the methods of the present invention involves a discrete wavelet transform on the raw data or selection of raw data to produce dwt coefficients. In one particular embodiment, the discrete wavelet transform is a 1D discrete wavelet transform. In a further preferred setting of the above embodiment, the 1D discrete wavelet transform is a 1D Daubechies wavelet transform.

In order to apply a discrete wavelet transform, a mother wavelet needs to be chosen. In a further preferred setting, the Daubechies wavelet transform uses a mother wavelet from the Daubechies family, most preferably being the DB8 mother wavelet.

In principle, in possible embodiments, any wavelet transform suitable for generating significant coefficients that capture information allowing discrimination at the single nucleotide level can be used in the method of the invention, such as the Daubechies DB4 wavelet, the Haar wavelet (which can also be considered part of the Daubechies family), least assymetric, coiflet, best localized. Alternative embodiments can use alternative algorithms to calculate the dwt including the lifting algorithm or the dual-tree complex wavelet transform. Other forms of discrete wavelet transform include the non- or undecimated wavelet transform (where downsampling is omitted), the Newland transform (where an orthonormal basis of wavelets is formed from appropriately constructed top-hat filters in frequency space). Wavelet packet transforms are also related to the discrete wavelet transform. Complex wavelet transform is yet another form.

In one step of the methods of the invention, the dwt coefficients are selected and analyzed. Typically, Scale and Wavelet coefficients that together provide a characteristic signature for the oligonucleotide mix under study are selected. The net result is a compact feature vector containing only coefficients significant for the task at hand and capturing a characteristic signature for the composition of the sample subject to the analysis using a computationally efficient algorithm. This feature vector is perfect input for machine learning techniques. Thus, data processing algorithms such as DWT will extract relevant features from the measurement data. The relevant features will be used as input features that will allow an input sample to be analyzed and classified by a machine-learning model such as neural networks, tree based models or support vector machines.

In the preferred embodiment a machine-learning model is to be used, the wavelet analysis (and filtered data reduction) method of the present invention extracts features and presents them as input for one (or more) of those machine-learning algorithms. In such embodiment, suitable reference samples with known composition are needed to train the classification algorithm before unknown samples can be successfully analysed.

Accordingly, current invention provides in a particular specific setting a method for analyzing melting curve raw data of nucleic acid from a test sample in which the step of performing discrete wavelet transform on the raw data to produce dwt coefficients results in the production of a compact feature vector containing dwt coefficients. Depending on the selection of the dwt coefficients, the compact feature vector will be a full or filtered compact feature vector containing dwt coefficients. This compact feature vector will in a further step be used to analyze the dwt coefficients and to classify the test sample based on the analysis.

In a preferred setting, the steps of analyzing and classifying is done by machine-learning models. Machine learning is concerned with the analysis of data, in particular it is concerned with algorithmically finding patterns and relationships in data, and using these to perform tasks such as classification and prediction in various domains. The machine learning model will hereto process the data contained in the feature vector and generate an output that classifies the test sample. Advantageously, the machine learning model has been configured through training to receive a compact feature vector generated from melting curve raw data and to process the data contained in the compact feature vector to generate outputs characterizing nucleic acid variation, such as SNP, single nucleotide insertion or deletion. In a particular preferred setting, the output will be associated with MSI and identify whether or not there is high amount of instability.

Thus, current invention further provides in a particular specific setting a method for analyzing melting curve raw data of nucleic acid from a test sample, the method comprising the steps of:
providing a source of nucleic acid from a subject;
amplifying said nucleic acid;
dissociating or associating the amplified nucleic acid to produce melting curve raw data;
optionally, performing data reduction on the raw data,
performing a discrete wavelet transform on the data to produce a full or filtered compact feature vector containing dwt coefficients; and
using the full or filtered compact feature vector as input for machine learning techniques.

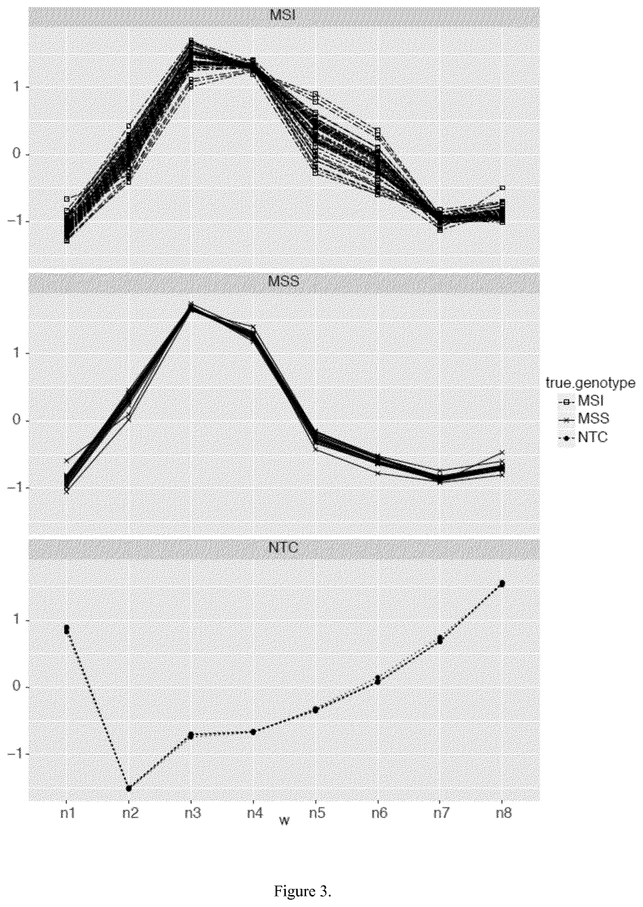

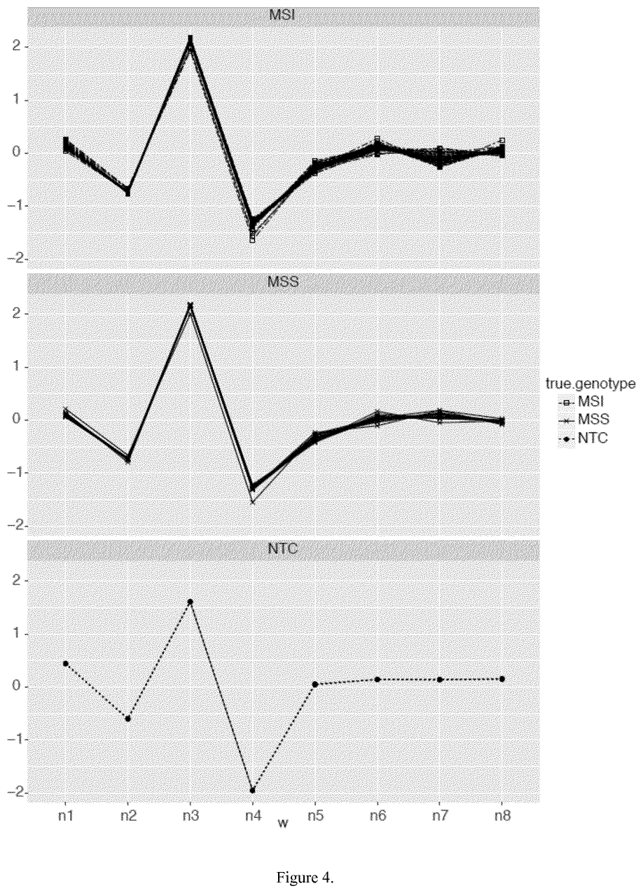

To that purpose, the method selects scale and wavelet coefficients that provide a characteristic signature to produce a full or filtered compact feature vector. Taking advantage of the wavelet transform, this selection of dwt coefficients allows to make a clear distinction between the patterns obtained for a wild type gene (FIG. 3B and FIG. 4B) and a mutant gene (FIG. 3A and FIG. 4A). Accordingly, the dwt coefficients are used to classify the test sample according to their nucleic acid composition.

The invention is particularly suitable for the combined analysis of several target molecules using multiple detection molecules in multiple reactions as current approach allows for the combined analysis of a patient or organism sample for several genes know to be implicated in a certain condition or phenotype. To that purpose, data defining a plurality of target molecules, each target having a respective label comprising characteristics of nucleic acid variation, are used. For such an implementation, the feature vectors obtained for each target molecule (measured in one or more experiments using one or more fluorophores) are then combined and fed into the machine learning algorithm as one. For such applications in particular, the compactness of the feature vector is a distinct advantage, allowing the application of powerful computing methods on the small embedded systems usually found in scientific instruments and medical devices.

The methods of the present invention are amendable to automation. Accordingly, the present invention also relates to a system that applies the described methods. Therefore, in a further embodiment, a method of the invention is provided wherein the steps of:
amplifying a nucleic acid obtained from a test sample;
dissociating or associating the amplified nucleic acid to produce melting curve raw data;
optionally, performing data reduction on the raw data;
performing discrete wavelet transform on the data to produce dwt coefficients;
performing the analysis of the dwt coefficients; and
classifying the test sample based on the analysis

are performed in an automated system.

Advantageously, said method is preceded by any of the following steps of:
liberating and/or isolating the nucleic acid potentially comprising the sequence from the source of a nucleic acid;
providing said liberated and/or purified nucleic acid potentially comprising the target to the step of amplifying said nucleic acid;

wherein at least the steps of:
liberating and/or isolating the nucleic acid potentially comprising the target homopolymeric repeat sequence from the source of a nucleic acid;
providing said liberated and/or purified nucleic acid potentially comprising the target sequence to the step of amplifying said nucleic acid;

are also performed in an automated system.

In a further, particularly advantageous and requiring minimal handling and technical preparation embodiment of the above embodiment, a method can be provided wherein at least steps of:
liberating and/or isolating the nucleic acid potentially comprising the target sequence from the source of a nucleic acid;
providing said liberated and/or purified nucleic acid potentially comprising the target sequence to the step of generating amplicons;
amplifying a nucleic acid sequence comprising the target sequence;
heating the amplified nucleic acid in the presence of a signal-generating oligonucleotide probe;
detecting the changes in the strength of said signal in the function of temperature to obtain at least one melting curve;

are performed in a cartridge engageable with said automated system.

In an automated system the method is carried out in an automated process, which means that the method or steps of the process are carried out with an apparatus or machine capable of operating with little or no external control or influence by a human being.

In a particular setting, the automated system consists of the following elements: an instrument, a console and cartridges. The instrument and console work in combination with the consumable cartridges. The instrument comprises control modules for performing assays. The console is a computer to control and monitor the instrument's actions and the cartridge status during the assays. In the cartridge the assay will be run, for example a real-time Polymerase Chain Reaction (PCR). After inserting a sample in a cartridge, pre-loaded with reagents, the cartridge is loaded into the instrument and the instrument controls the assay which is performed autonomously in the cartridge. After the assay has run, the console software processes the results and generates a report accessible for the end-user of the automated system.

The automated system can be an open or a closed, automated system. When a sample has been added or inserted in the cartridge-based system, the cartridge-based system is closed and stays closed during the operation of the system. The closed system contains all the necessary reagents on board, so the closed configuration provides the advantage that the system performs contamination-free detection. Alternatively, an open, accessible cartridge can be used in an automated system. The necessary reagents are added in the open cartridge as required, thereafter a sample can be inserted in the open cartridge and the cartridge can be run in a closed, automated system.

Preferably, cartridge-based systems containing one or more reaction chambers and one or more fluid chambers are used. Some of the fluid chambers may hold fluid which is used for producing a lysate from the sample. Other chambers may hold fluids such as reaction buffers, washing fluids and amplification solutions. The reaction chambers are used to perform the different steps of the detection such as washing, lysis and amplification.

As used herein, the term “cartridge” is to be understood as a self-contained assembly of chambers and/or channels, which is formed as a single object that can be transferred or moved as one fitting inside or outside of a larger instrument suitable for accepting or connecting to such cartridge. Some parts contained in the cartridge may be firmly connected whereas others may be flexibly connected and movable with respect to other components of the cartridge. Analogously, as used herein the term “fluidic cartridge” shall be understood as a cartridge including at least one chamber or channel suitable for treating, processing, discharging, or analyzing a fluid, preferably a liquid. An example of such cartridge is given in WO2007004103. Advantageously, a fluidic cartridge can be a microfluidic cartridge. In the context of fluidic cartridges the terms “downstream” and “upstream” can be defined as relating to the direction in which fluids flow in such cartridge. Namely, a section of a fluidic path in a cartridge from which a fluid flows towards a second section in the same cartridge is to be interpreted as positioned upstream of the latter. Analogously, the section to which a fluid arrives later is positioned downstream with respect to a section which said fluid passed earlier.

In general, as used herein the terms “fluidic” or sometimes “microfluidic” refers to systems and arrangements dealing with the behavior, control, and manipulation of fluids that are geometrically constrained to a small, typically sub-millimeter-scale in at least one or two dimensions (e.g. width and height or a channel). Such small-volume fluids are moved, mixed, separated or otherwise processed at micro scale requiring small size and low energy consumption. Microfluidic systems include structures such as micro pneumatic systems (pressure sources, liquid pumps, micro valves, etc.) and microfluidic structures for the handling of micro, nano- and picoliter volumes (microfluidic channels, etc.). Exemplary fluidic systems were described in EP1896180, EP1904234, and EP2419705 and can accordingly be applied in certain embodiments of the presented herein invention.

Melting curve data can be obtained from samples containing appropriate fluorescent moieties processed by any instrument or method for conducting amplification such as thermal cycling, PCR, quantitative PCR or similar processing. Melting curve data can be obtained from any fluorometric or spectrophotometric apparatus equipped with a means of adjusting the sample temperature to above the melting temperature of the DNA sample. Examples of such instruments include, but are not limited to, thermal cyclers (both modular and multi-block), optical thermocyclers commonly used for quantitative PCR, fluorometers with temperature control, PCR machines, batch heaters or chillers, and other similar instruments, all of which are equipped with associated optics so as to permit generation and maintenance of specific temperatures for a defined period of time while measuring fluorescence. Those skilled in the art will recognize other instruments or methods known in the art used in connection with the generation of melting curve data are within the spirit and scope of the present invention.

In a particularly desired embodiment in accordance with the above-listed embodiments, to streamline and facilitate the interpretation of the results of the method according to present invention, the analysis on the melting curve is also performed in an automated manner by means of a computer-implemented method.

Embodiments of the methods described herein are also embodiments of the computer-implemented methods described herein. Technical effects obtained by the methods described herein are also technical effects obtained by the computer-implemented methods described herein. The computer-implemented methods herein are particularly suitable for classifying test samples involving the combined analysis of several target multiplexing experiments generating large raw datasets requiring discrimination of subtle but molecularly significant differences. The computer-implemented methods herein are particularly suitable for the combined analysis of several target molecules using multiple detection molecules in multiple reactions as current approach allows for the combined analysis of a patient or organism sample for several genes know to be implicated in a certain condition or phenotype. For such an implementation, the feature vectors obtained for each target molecule (measured in one or more experiments using one or more fluorophores) are then combined and fed into the machine learning algorithm as one. For such applications in particular, the compactness of the feature vector is a distinct advantage, allowing the application of powerful computing methods on the small embedded systems usually found in scientific instruments and medical devices.

Thus, another aspect relates to a computer-implemented method for obtaining and transforming melting curve raw metrics of nucleic acid from a test sample, the method comprising the steps of:
producing melting curve raw data from a nucleic acid;
performing discrete wavelet transform on the data to produce dwt coefficients;
select those coefficients identified as the most relevant for melting curve analysis of said nucleic acid;
performing the analysis of the dwt coefficients; and
classifying the test sample based on the analysis.

Advantageously, the steps of analyzing and classifying is done by machine-learning models that generate outputs characterizing nucleic acid variation, such as SNP, single nucleotide insertion or deletion. In a particular preferred setting, the output will be associated with MSI and identify whether or not there is high amount of instability. To that purpose, the methods of the invention will typically include a step of data visualization. Data visualisation communicates the complex information in a way that is easier to interpret by turning data into visually engaging images, colors, stories, etc. . . . . Such visualization may with e.g. the use of color codes help to simply and rapidly identify nucleotide variations in amplified nucleic acid based on wavelet transform output graphs.

In some embodiments the step of producing melting curve raw data from a nucleic acid in the computer-implemented method comprises the steps of:
providing a source of nucleic acid from a subject,
amplifying said nucleic acid, and
dissociating or associating the amplified nucleic acid, to produce melting curve raw data.

In some embodiments, said computer-implemented method is preceded by any of the following steps of:
liberating and/or isolating the nucleic acid potentially comprising the target sequence from the source of a nucleic acid; and/or
providing said liberated and/or purified nucleic acid potentially comprising the target to the step of amplifying said nucleic acid.

The invention further relates to a data processing device comprising means for carrying out the computer-implemented method for obtaining and transforming melting curve raw metrics of nucleic acid from a test sample. The invention further relates to the data processing device in combination with and/or coupled to means for producing melting curve raw data from a nucleic acid, optionally in combination with and/or coupled to means for liberating and/or isolating the nucleic acid potentially comprising the target sequence from the source of a nucleic acid.

The means for producing melting curve raw data from a nucleic acid may comprise one or more of the following:
means for providing a source of nucleic acid from a subject;
means for amplifying said nucleic acid; and
means for dissociating or associating the amplified nucleic acid.

The means for liberating and/or isolating the nucleic acid potentially comprising the target sequence from the source of a nucleic acid may comprise a cartridge engageable with the data processing device.

The invention also relates to a computer program comprising instructions which, when the program is executed by a computer (optionally coupled to one or more additional means), cause the computer to carry out the computer-implemented method for obtaining and transforming melting curve raw metrics of nucleic acid from a test sample.

The invention also relates to a computer-readable medium comprising instructions which, when executed by a computer (optionally coupled to one or more additional means), cause the computer to carry out the computer-implemented method for obtaining and transforming melting curve raw metrics of nucleic acid from a test sample.

The following examples are provided to aid the understanding of the present invention, the true scope of which is set forth in the appended claims.

### EXAMPLES

### Example 1. Molecular Beacon Melting Curve for the SEC31A MSI Marker in Cancer Patient Samples

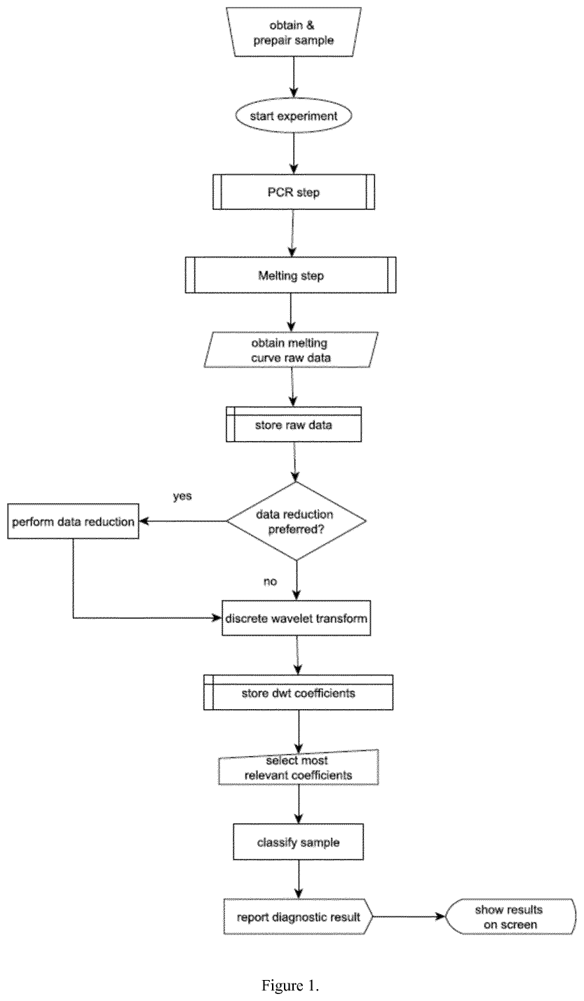

Very minor changes of 1 nucleotide in length in a homopolymeric nucleotide repeat sequence in the human SEC31A marker, positioned at chr4:82864395 and containing a homopolymeric repeat of 9 adenines (A).were assessed according to the flow chart represented in FIG. 1.

The wild-type (WT) homopolymeric repeat sequence (bolded and underlined) and its specific surrounding sequence of SEC31A is given below:

(SEQ ID NO. 1)
CAACTTCAGCAGGCTGTAGTCTGAGAAGCATCAATTTTCAACTTCAGCAG
GCTGTGCAGTCACAAGGATTTATCAATTATTGCCAAAAAAAAATTGATGC
TTCTCAGACT.

To detect the nucleotide changes in the repeat sequence of SEC31A, a molecular beacon detection probe was designed having the sequence of

(SEQ ID NO. 2)
CGCACTTGCCAAAAAAAATTGATGGTGCGTAAA

and was labeled with Atto647 as a fluorescently labeling molecule, whereas BHQ2 was used as a quencher (stem region of the molecular beacon probe is indicated in italics, the probe hybridizing region is bolded wherein the repeat sequence identical to mutated SEC31A marker comprising 8 adenine repeats instead of 9 is bolded and underlined).

FFPE samples from colorectal cancer patients were provided into Biocartis Idylla™ fluidic cartridges. The cartridges were closed and loaded onto the Biocartis Idylla™ platform for automated PCR-based genetic analyses, after which the automated sample processing was initiated. Firstly, the patients' DNA was released from the FFPE samples and then pumped into PCR compartments of the cartridges. Next, asymmetric PCR amplification of the region surrounding the SEC31A homopolymer repeat sequence was performed in each cartridge using the following primers

FWD: 5′-CAACTTCAGCAGGCTGT-3′ (SEQ ID NO. 3) and REV: 5′-AGTCTGAGAAGCATCAATTTT-3′ (SEQ ID NO. 4). The PCR amplification was performed in the presence of the above-described SEC31A,-specific molecular beacon probe.

After the PCR, the PCR products were denatured in the cartridges for 2 min at 95° C. and then cooled down to 45° C. for 1 min to allow sufficient time for the hybridization of the SEC31A-specific molecular beacon probe to its targets. Next, a melting curve analysis was performed while still on Idylla system by heating the mixture from 40° C. to 76.6° C. in steps of 0.3° C. (12 s per cycle) and at the same time monitoring the fluorescence signals (approx. 8s per cycle) after every 0.3° C. increase providing the melting curve raw data (further referred to as ‘X’).

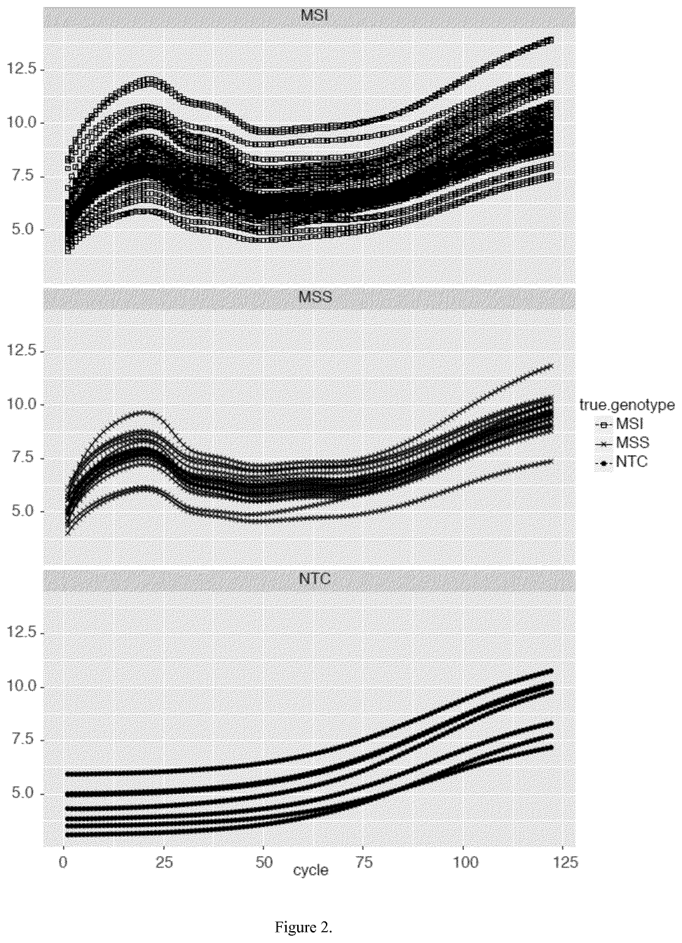

FIG. 2 shows the resulting fluorescence signal measurements obtained from several reference samples in function of temperature for SEC31A. FIG. 2A shows melting profiles representing samples that are characterized as 20% mutated+80% WT (MSI). FIG. 2B indicates melting profiles representing samples that are characterized as 100% WT (MSS). FIG. 2C shows melting profiles representing samples that are characterized as empty sample (NTC).

### Example 2. Wavelet Transform Curve for the SEC31A MSI Marker in Cancer Patient Samples

A package of functions for computing wavelet filters, wavelet transforms and multiresolution analyses (Aldrich, 2015) was applied to compute the discrete wavelet transform coefficients for a univariate or multivariate time series representing the raw melting curve data (X). The raw melting curve data were obtained from 317 patient samples. A first implementation has been build using the R program (https://www.r-project.org/) augmented with the wavelets package of Aldrich, 2015. For the present SEC31A experiment, a one dimensional wavelet transform was applied using the DB8 mother wavelet. The discrete wavelet transform was computed via the pyramid algorithm, based on the pseudocode written by Percival and Walden (2000), pp. 100-101. When the boundary setting is put on “periodic” the resulting wavelet and scaling coefficients are computed without making changes to the original series, in such case the pyramid algorithm treats X as if it is circular. However, when the boundary setting is put on “reflection” a call is made to extend series, resulting in a new series which is reflected to twice the length of the original series. The wavelet and scaling coefficients are then computed by using a periodic boundary condition on the reflected series resulting in twice as many wavelet and scaling coefficients at each level. Several levels of decomposition can be applied. Figures display wavelet coefficients in the third level of decomposition.

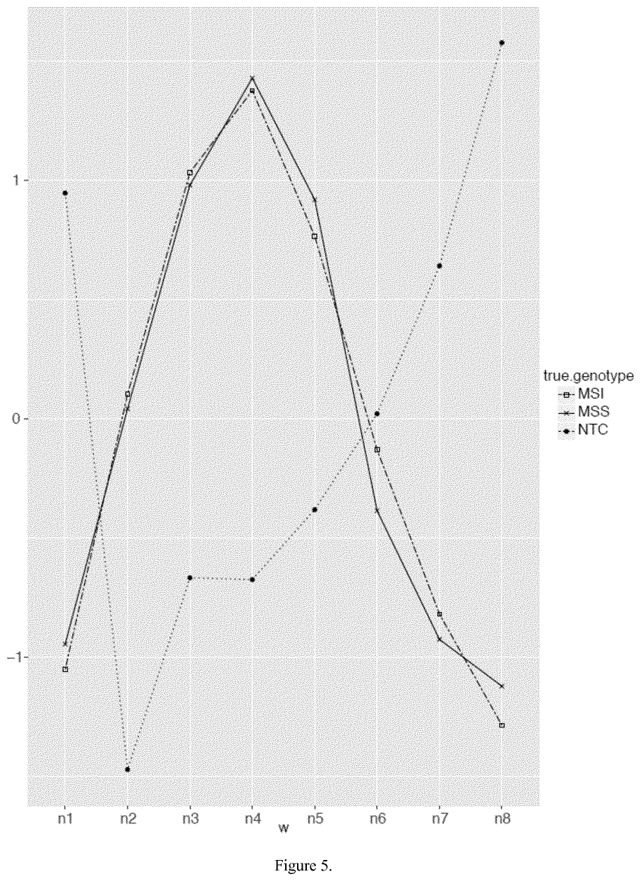

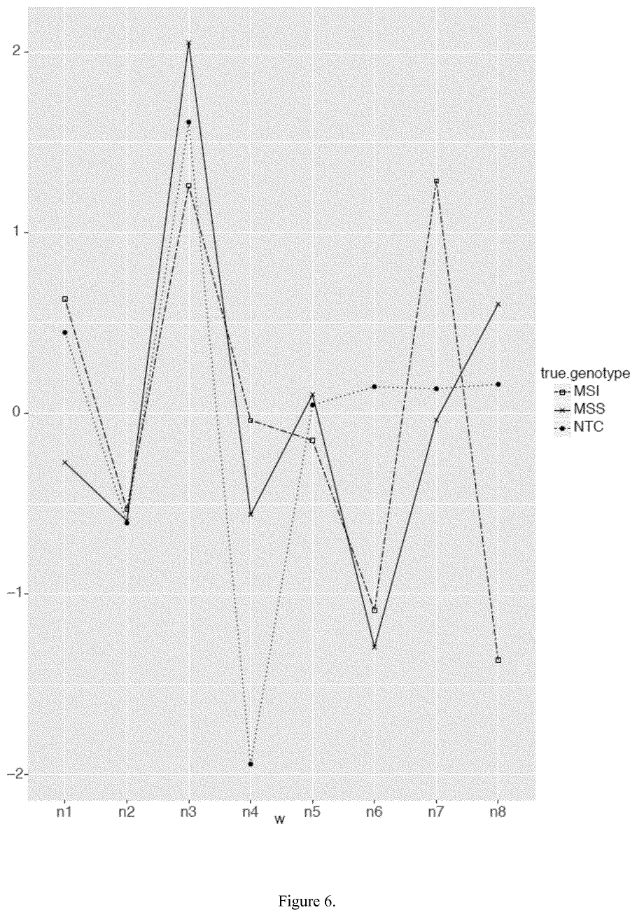

For the present experiment, periodic boundary conditions were shown to be adequate. The graph in FIG. 3 represents wavelet transformed values for the SEC31A gene in 317 patient samples using the scale function from Daubechies DB8. The graph in FIG. 4 represents wavelet transformed values for the SEC31A gene in the same patient samples using the wavelet function from Daubechies DB8. As can be derived from FIGS. 3 and 4, based on the plotted wavelet transformation values, a clear distinction can be made between the patterns obtained for a wild type gene (FIG. 3B and FIG. 4B) and a mutant gene (FIG. 3A and FIG. 4A). The graphs in FIGS. 5 and 6 represent a direct comparison of the wavelet transformed patterns for the SEC31A gene showing one pattern for each sample class (wild type, mutant and NTC) using the scale function and the wavelet function form Daubechies DB8 respectively.

### Example 3. Wavelet Transform Curve for Several MSI Markers in Cancer Patient Samples

Using multiplexing techniques and several concurrent reactions, WT or mutant status are obtained for several genes known to be involved in colorectal cancer. In a further experiment, the MSI status for seven genes using two duplexes and three singleplexes was determined.

### Example 4. SEC31A MSI Marker Classification in Cancer Patient Samples

The obtained wavelet and scale coefficients as described in Examples 1 to 3 were subsequently used as input to a neural net for classification. The resulting data vectors of the DWT are sampled for the most distinguishing levels of decomposition. The scale vector is scaled and centered around zero to ensure that the value distributions are comparable to those of the wavelet coefficients. This allows for the use of one feature vector per observation compiled from both sets of coefficients. This improves classification by the machine learning algorithms.

Definition and training of the neural net used the Tensorflow software package. Program input, program output and program user interface were provided using the R program as described in example 2. The Keras package was used to integrate Tensorflow functionality with R.

In a first setting, the R-Keras-Tensorflow system was used both for training the neural net using reference samples and for classification of unknown samples. This implementation is operational since Mar. 15, 2017.

In a second setting, the R-Keras-Tensorflow system was used for training of the neural net and the resulting code for classification of unknown samples was integrated in the Biocartis Idylla™ platform and allowed for automated processing & classification of unknown samples.

### Example 5. Wavelet Transform Curve for the SEC31A MSI Marker in Cancer Patient Samples Using Other Wavelet Filters

Next to the preferred embodiment, other mother wavelets can also be applied to obtain useful transformed measurement data. In this example DB4 and Haar mother wavelets are performed on the same dataset as used in example 2.

A package of functions for computing wavelet filters, wavelet transforms and multiresolution analyses (Aldrich, 2015) was applied to compute the discrete wavelet transform coefficients for a univariate or multivariate time series representing the raw melting curve data (X). The raw melting curve data were obtained from 317 patient samples. A first implementation has been build using the R program (https://www.r-project.org/) augmented with the wavelets package of Aldrich, 2015.

For the present SEC31A experiment, a one dimensional wavelet transform was applied using the DB4 and Haar mother wavelets. The discrete wavelet transform was computed via the pyramid algorithm, based on the pseudocode written by Percival and Walden (2000), pp. 100-101. When the boundary setting is put on “periodic” the resulting wavelet and scaling coefficients are computed without making changes to the original series, in such case the pyramid algorithm treats X as if it is circular. However, when the boundary setting is put on “reflection” a call is made to extend series, resulting in a new series which is reflected to twice the length of the original series. The wavelet and scaling coefficients are then computed by using a periodic boundary condition on the reflected series resulting in twice as many wavelet and scaling coefficients at each level. Several levels of decomposition can be applied. Figures display wavelet coefficients in the third level of decomposition.

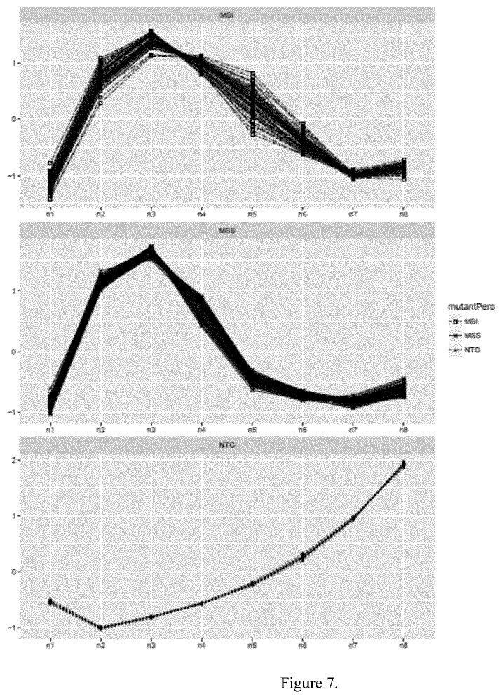

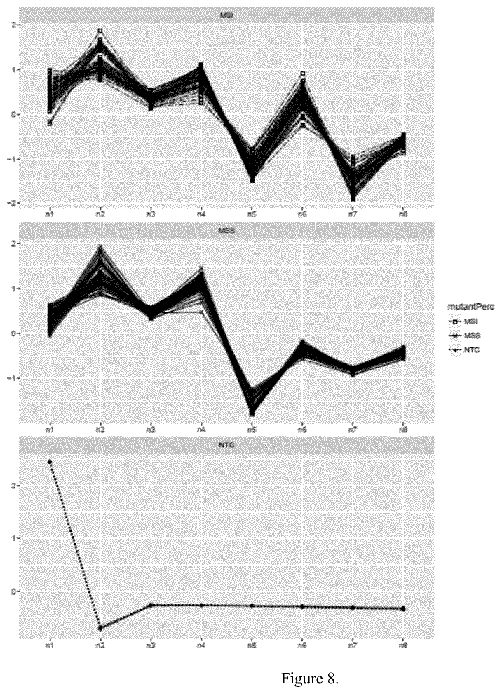

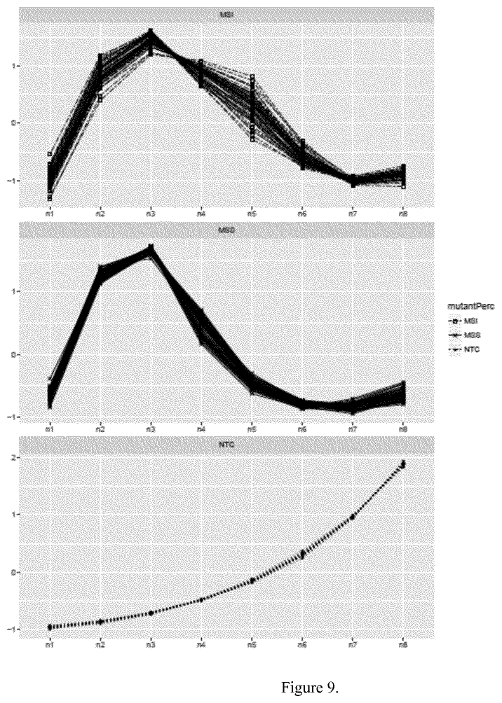

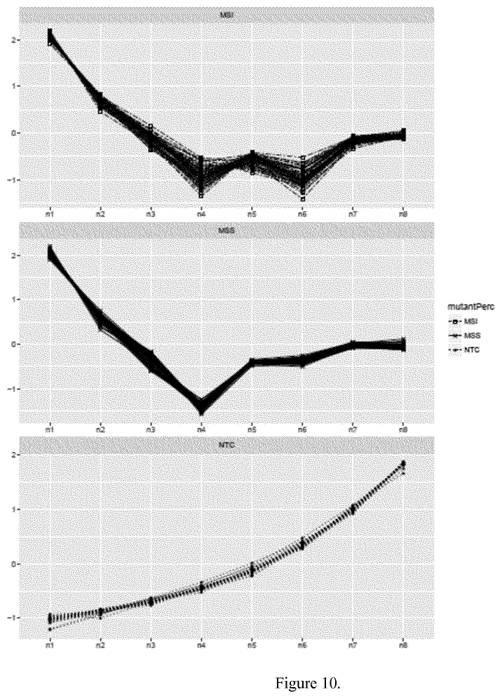

For the present experiment, periodic boundary conditions were shown to be adequate. The graph in FIG. 7 represents wavelet transformed values for the SEC31A gene in 317 patient samples using the scale function from Daubechies DB4. The graph in FIG. 8 represents wavelet transformed values for the SEC31A gene in the same patient samples using the wavelet function from Daubechies DB4. The graph in FIG. 9 represents wavelet transformed values for the SEC31A gene in 317 patient samples using the scale function from the Haar wavelet. The graph in FIG. 10 represents wavelet transformed values for the SEC31A gene in the same patient samples using the wavelet function from the Haar wavelet. As can be derived from FIGS. 7, 8, 9 and 10, based on the plotted wavelet transformation values, clear distinctions can be made between the patterns obtained for a wild type gene (FIG. 7B and FIG. 8B) and a mutant gene (FIG. 7A and FIG. 8A) for Daubechies DB4 as well as between the patterns obtained for a wild type gene (FIG. 9B and FIG. 10B) and a mutant gene (FIG. 9A and FIG. 10A) for the Haar wavelet.

### REFERENCES

Athamanolap, P. et al. Trainable High Resolution Melt Curve Machine Learning Classifier for Large-Scale Reliable Genotyping of Sequence Variants. PLOS ONE 9, e109094 (2014).
Cohen A., Daubechies I., and P. Vial, Wavelets on the interval and fast wavelet transforms, Applied Comput. Harmon. Anal., vol. 1, 1993, pp. 54-81.
Daubechies, I. (1992) Ten lectures on wavelets. Society for Industrial and Applied Mathematics Gray, R. D. & Chaires, J. B. Analysis of Multidimensional G-Quadruplex Melting Curves. Curr. Protoc. Nucleic Acid Chem. Chapter Unit 17.4 (2011).
Liao, Y. et al. Simultaneous Detection, Genotyping, and Quantification of Human Papillomaviruses by Multicolor Real-Time PCR and Melting Curve Analysis. J. Clin. Microbiol. 51, 429-435 (2013).
Palais, R. & Wittwer, C. T. Mathematical algorithms for high-resolution DNA melting analysis. Methods Enzymol. 454, 323-343 (2009).
Percival, D. B. and Walden A. T. (2000) Wavelet Methods for Time Series Analysis, Cambridge University Press.
R. L. de Queiroz, Subband processing of finite length signals without border distortions, in Proc. IEEE Int. Conf Acoust., Speech, Signal Processing, Vol. IV, 1992, pp. 613-616.
Ramezanzadeh, M., Salehi, M. & Salehi, R. Assessment of high resolution melt analysis feasibility for evaluation of beta-globin gene mutations as a reproducible, cost-efficient and fast alternative to the present conventional method. Adv. Biomed. Res. 5, 71 (2016).
Reed, G. H., Kent, J. O. & Wittwer, C. T. High-resolution DNA melting analysis for simple and efficient molecular diagnostics. Pharmacogenomics 8, 597-608 (2007).
Williams J. R. and Amaratunga K., A discrete wavelet transform without edge effects using wavelet extrapolation, J. Fourier Anal. Appl., Vol. 3, No. 4, 1997, pp. 435-449.
Wittwer, C. T. High-resolution DNA melting analysis: Advancements and limitations. Hum. Mutat. 30, 857-859 (2009).

1. A method for analyzing melting curve raw data of nucleic acid from a test sample, the method comprising the steps of:
producing melting curve raw data from a nucleic acid;
performing discrete wavelet transform on the raw data to produce discrete wavelet transform coefficients, further referred to as dwt coefficients;
performing the analysis of the dwt coefficients;
and classifying the test sample based on the analysis.

2. Method according to claim 1, wherein the melting curve raw data are obtained from nucleic acids with one or more SNPs or nucleic acids with length variations, preferably being insertions or deletions.

3. Method according to claim 1, wherein the melting curve raw data are obtained from nucleic acids with subtle microsatellite changes, preferably being nucleic acids with homopolymeric repeat sequence variations.

4. Method according to claim 1, wherein the nucleic acid is an amplified nucleic acid.

5. Method according to claim 1, wherein melting curve raw data are produced by dissociating the amplified nucleic acid in the presence of a dye-labeled nucleic acid, preferably a dye-labeled beacon probe.

6. Method according to claim 1, wherein the step of producing melting curve raw data from a nucleic acid is followed by a step of performing data reduction on the raw data to generate a selection of raw data, and wherein the discrete wavelet transform is performed on the selection of raw data.

7. Method according to claim 1, wherein the step of performing the analysis of the dwt coefficients comprises selecting those dwt coefficients identified as the most relevant and performing the analysis of the selected dwt coefficients.

8. Method according to claim 1, wherein the discrete wavelet transform is a one-dimension wavelet transform.

9. Method according to claim 1, wherein the discrete wavelet transform uses a mother wavelet from the Daubechies family, preferably being the DB8 wavelet.

10. Method according to claim 1, wherein the classifying is a genotyping record and one or more visual displays of the genotyping.

11. Method according to claim 1, wherein the steps of:
producing melting curve raw data from a nucleic acid;
optionally, performing data reduction on the raw data to generate a selection of raw data;
performing discrete wavelet transform on the raw data or selection of raw data produce discrete wavelet transform coefficients, further referred to as dwt coefficients;
performing the analysis of the dwt coefficients,
wherein the performing the analysis of the dwt coefficients optionally comprises selecting those dwt coefficients identified as the most relevant and performing the analysis of the selected dwt coefficients; and
classifying the test sample based on the analysis are performed in an automated system.

12. Method according to claim 7 wherein the method is a computer-implemented method.

13. A data processing device comprising means for carrying out the computer-implemented method according to claim 12.

14. A computer program comprising instructions which, when the program is executed by a computer, cause the computer to carry out the computer-implemented method according to claim 12.

15. A computer-readable medium comprising instructions which, when executed by a computer, cause the computer to carry out the computer-implemented method according to claim 12.
